(19) United States
(12) Patent Application Publication
(10) Pub. No.: US 2011/0290246 A1
(43) Pub. Date: Dec. 1, 2011

(54) Method and apparatus for protection of trachea during ventilation

(76) Inventor: Oron ZACHAR, Tel Aviv (IL)

(21) Appl. No.: 13/095,361

(22) Filed: Apr. 27, 2011

Related U.S. Application Data
(63) Continuation-in-part of application No. 12/823,114, filed on Jun. 24, 2010.
Application No. 12/823,114 is a continuation-in-part of application No. PCT/US2010/039881, filed on Jun. 24, 2010.
Application No. PCT/US2010/039881 is a continuation-in-part of application No. PCT/US2009/062227, filed on Oct. 27, 2009.
(60) Provisional application No. 61/350,913, filed on Jun. 2, 2010.
(60) Provisional application No. 61/329,106, filed on Apr. 29, 2010.

(30) Foreign Application Priority Data
Jun. 23, 2010 (GB) 1010564.1

(51) Int. Cl. A61M 16/04

(52) U.S. Cl. 128/200.26; 128/207.15

(57) ABSTRACT

Disclosed are methods for intubating a patient, comprising deploying a tracheal tube, a sleeve and a cuff into a human trachea such that after deployment, the tracheal tube passes through the sleeve within the trachea, the cuff contacts the outer surface of the tracheal tube and the inner surface of the sleeve and spaces the sleeve from the tube, and the outer surface of the sleeve contacts the trachea, so as to provide a seal, in the interstitial area between the wall of the trachea and the tube, between a proximal portion of the human trachea above the cuff and a distal portion of the human trachea below the cuff. Other embodiments are also disclosed.

### RELATED APPLICATION INFORMATION

This application is a continuation in part of U.S. Non-Provisional application Ser. No. 12/823,114 filed on Jun. 24, 2010 and incorporated herein by reference in it entirety. This application is also a continuation in part of PCT/US2010/039881 filed on Jun. 24, 2010 and incorporated herein by reference in it entirety. This application is also a continuation in part of PCT/US2009/062227 filed on Oct. 27, 2009 and incorporated herein by reference in it entirety. This application claims priority from U.S. Provisional Application Ser. Nos. 61/108,594, filed Oct. 27, 2008 and incorporated herein by reference in it entirety; 61/219,769, filed Jun. 24, 2009 and incorporated herein by reference in it entirety; 61/236,553, filed Aug. 25, 2009 and incorporated herein by reference in it entirety; 61/238,151, filed Aug. 29, 2009 and incorporated herein by reference in it entirety; 61/329,106 filed on Apr. 29, 2010 and incorporated herein by reference in it entirety; and 61/350,913 filed on Jun. 2, 2010 and incorporated herein by reference in it entirety. This application claims priority from GB 1010564.1 filed on Jun. 23, 2010 and incorporated herein by reference in it entirety. The contents of all of the aforementioned applications are incorporated herein by reference.

### BACKGROUND

During administration of anesthesia to a patient, or in situations in which the patient is in the intensive care unit (ICU), it is standard practice to intubate the patient with a tube, introduced into the trachea, to facilitate pulmonary ventilation. The adult human trachea is approximately 2.0-2.5 cm across and approximately 10-16 cm in length (from the larynx to the carina). The present state of the art of intubation is illustrated in FIG. 1, which is taken from FIG. 2A of U.S. Pat. No. 6,843,250. As shown in FIG. 1, an endotracheal tube (ETT) 100 which is inserted into a patient airway 111 typically includes an inflatable balloon or cuff 112 near its distal end. The cuff 112, when inflated, performs a dual function: (a) the cuff occludes the air passageway, thus establishing a closed system whereby the gas pressure in the airway distal to the inflated cuff can be maintained at a desired level, thus providing means of controlling the exchange of blood gases in lungs. (b) the cuff provides a barrier against inflow of aspirated gastric contents or other matter foreign to the lungs.

Unfortunately, there are several complications often associated with such intubation. First, 8-28% of intubated patients develop ventilation associated pneumonia (VAP); there is 20-30% mortality rate among VAP patients. It is estimated that in the USA, VAP and associated complications increase patient time in intensive care units by about 4-6 days, at a cost increase of $20,000 to $40,000. VAP occurs because the insertion of the ETT bypasses the protective system of the tracheo-bronchial tree. Secretions, mucuous or aspirated gastric material, which normally would be directed harmlessly through the digestive system, follow the path of the tube into the airways. Although the use of the balloon or cuff is supposed to prevent such fluids from entering the lungs, the cuff is not infallible. Inter alia, the inflatable cuff, on a typical commercially available endotracheal tube, is in the form of an oval-shaped balloon. The oval-shaped balloon permits these secretions to pool around the surface of the balloon proximal to the oral cavity, particularly in the vicinity of the region where the balloon contacts the tracheal wall; sometimes these fluids pass by the balloon and into the tracheo-bronchial tree. This passage of unwanted fluids past the inflated cuff of the tracheal tube device is thought to be due to the patient's breathing cycle producing fluctuating inhalation/exhalation pressures on the downstream ovate surface of the inflated cuff, causing the cuff and/or the tracheal conduit to act somewhat in the manner of a peristaltic pump.

While it is standard protocol to attempt to suction the region in which fluid tends to collect, suctioning is awkward and, done blindly, may result in the insufficient suctioning of the pharynx. In advanced tube designs, the tube device is provided not only with an inflation line to the cuff but also with a suction line opening to a region above the cuff. In practice however, due to the finite axial length accommodated by the tape or other fastening means required to attach the cuff sealingly to the main tube of the structure, the opening from the suction line is disposed too far above the upstream ovate surface of the cuff to ensure removal by suction of all the unwanted fluids collecting in that region. Moreover, the oval shape of the cuff inevitably leads to having the most crucial area of fluid collection, at the contact between the cuff and the trachea surface, being too narrow for the reach of any suction device. Hence, suction above oval balloons may not ensure complete removal of all secretions.

Furthermore, when the cuff is deflated for removal, fluid collected on the upper surface of the cuff, proximal to the oral cavity, may flow into the lungs.

We believe the state of the art with regard to presently-used cuffs and the associated suction devices is represented by U.S. Pat. Nos. 4,979,505, 5,520,175, 5,937,861, 6,062,223, 7,089,942, and 7,293,561 and US patent publications nos. 20030024534 and 20080115789, and references therein. These patents and patent publications, as well as all other publications mentioned herein, are incorporated herein by reference.

A second major set of complications arising from tracheal intubation is associated with the cuff sealing pressure. To prevent the leakage of air from between the inflated cuff and the tracheal wall during mechanical ventilation, the pressure in the cuff must be equal to or greater than the peak inspiratory pressure within the airway. Peak inspiratory pressures are only achieved for 10%-25% of the ventilatory cycle but may be as high as 50 mm of mercury. Since the pressure within the standard cuff is static, to achieve continuous good sealing the cuff pressure must ideally be maintained at this relatively high pressure (equal to or greater than peak airway pressure) throughout the ventilatory cycle, to prevent leaks during the highest pressure portion of the cycle. Yet such sealing pressure cannot be implemented in practice due to the risk of tissue anoxia and other complications: as the cuff pressure exceeds the capillary pressure of the tracheal tissues (which is normally 25 mm of mercury), tissue anoxia occurs, and varying degrees of tracheal injury result. The injuries range from mild erosion of the mucosa, to destruction of the tracheal cartilage rings, to segmental tracheomalacia with dilatation of the trachea. More dramatic is full thickness erosion, with perforation of the inominate artery anteriorly or posteriorly into the esophagus; both of these events are associated with a high rate of mortality. Late complications of tracheal stenosis, from mild to incapacitating obstruction, are most often observed in patients requiring long-term ventilatory support, such as patients hospitalized in the ICU.

Most tracheal tubes currently in use employ a soft cuff that, when inflated, assumes a fusiform shape presenting a narrow surface in contact with the trachea mucosa. Any prolonged pressure above 25 torr increases the risk of tracheal necrosis. The state of the art in dealing with excess cuff pressure is described in U.S. Pat. No. 5,937,861.

In addition to endotracheal tubes, tracheal stents are known in the art. Unlike ETT's, however, which are generally used to control a patient's breathing, tracheal stents are merely used to keep the air passage open. The state of the art of tracheal stents is exemplified in www.emedicine.com/ent/topic593.htm and in Ann. Thorac. Med. (2006) 1:92-7, US patent publication 20030024534, PCT patent publication WO 2004/067060 A2, and references therein. The general art of stent construction and design is further exemplified in U.S. Pat. Nos. 7,291,166, 7,300,459, 7,374,570, 7,285,132, 6,821,291, 6,458,152, and 5,716,410, US patent publication no. 20080077222, and references therein.

### BRIEF STATEMENT OF THE INVENTION

There is provided, in accordance with an embodiment of the invention, a method for tracheal intubation in a patient in need thereof, comprising aligning, in the trachea of the patient, (a) a tracheal tube having a distal end which is inserted into the trachea and a proximal end which remains outside the trachea; (b) a sleeve through which the tracheal tube passes, the sleeve having an inner surface which faces the tracheal tube and an outer surface and proximal and distal ends which correspond to the proximal and distal ends, respectively, of the tracheal tube, the sleeve being of a length less than the distance between the larynx and the carina; and (c) a cuff which contacts at least a portion of the outer surface of the tracheal tube and at least a portion of the inner surface of the sleeve in a manner which substantially creates a seal between the distal and proximal ends of the inner surface of the sleeve and the distal and proximal ends of the outer surface of the tracheal tube, whereby to radially space the tracheal tube from the sleeve. In some embodiments, the outer surface of the sleeve contacts the wall of the trachea. In some embodiments, the tube is inserted into the trachea before the sleeve is inserted into the trachea. In some embodiments, the tube is inserted into the trachea after the sleeve is inserted into the trachea. In some embodiments, the sleeve and the tube are inserted concomitantly into the trachea.

There is also provided, in accordance with an embodiment of the invention, a method of reducing the likelihood of fluids leaking from the trachea of a patient undergoing intubation into a lung theorof, the method comprising deploying a tracheal tube, a sleeve and a cuff into a human trachea such that after deployment, the tracheal tube passes through the sleeve within the trachea, the cuff contacts the outer surface of the tracheal tube and the inner surface of the sleeve and spaces the sleeve from the tube, and the outer surface of the sleeve contacts the trachea, so as to provide a seal, in the interstitial area between the wall of the trachea and the tube, between a proximal portion of the human trachea above the cuff and a distal portion of the human trachea below the cuff. In some embodiments, the cuff is an expandable cuff, the sleeve is longitudinally rigid and radially expandable along at least a portion thereof, and the deploying includes: (a) simultaneously inserting the tracheal tube, the expandable cuff and the sleeve into the trachea; and (b) after the inserting, inflating the inflatable cuff, so as to cause radial expansion of the sleeve and create said seal by contacting the sleeve to an inner surface of the trachea and by sealingly contacting the inner surface of the sleeve and the outer surface of the tube. In some embodiments, the cuff is an expandable cuff, the sleeve is longitudinally rigid and radially expandable along at least a portion thereof, and the deploying includes: (a) emplacing the sleeve in the trachea of the patient; (b) thereafter inserting the tube and the cuff into trachea such that the tube passes through the sleeve with cuff positioned between the tube and sleeve; and (c) thereafter expanding the cuff so that the outer surface of the sleeve contacts the wall of the trachea, the cuff contacts the inner surface of the sleeve and the outer surface of the tube and spaces the sleeve from the tube, whereby to provide said seal. In some embodiments, the cuff is an expandable cuff, the sleeve is longitudinally rigid and radially expandable along at least a portion thereof, and the deploying includes: (a) inserting the tube and the cuff into trachea; (b) emplacing the sleeve in the trachea of the patient such that the tube passes through the sleeve with cuff positioned between the tube and sleeve; and (c) thereafter expanding the cuff so that the outer surface of the sleeve contacts the wall of the trachea, the cuff contacts the inner surface of the sleeve and the outer surface of the tube and spaces the sleeve from the tube, whereby to provide said seal.

In some embodiments, the sleeve is sized and shaped to contact the tracheal wall. In some embodiments, the sleeve has a substantially circular cross-section; in some embodiments, the maximum diameter of the sleeve is not more than about 2.5 cm. In some embodiments, the sleeve has a non-circular cross section; in some embodiments, the sleeve is sized and shaped to contact the tracheal wall in a manner which substantially seals the lungs from the pharynx. In some embodiments, the sleeve is of a length that fits between the larynx and the carina. In some embodiments, the sleeve has a length of from 2 to 8 cm. In some embodiments, the distal end of the sleeve is positioned 2 to 6 cm above the carina.

In some embodiments, the sleeve is substantially rigid in its axial direction along at least 50% of its length. In some embodiments, the sleeve is substantially rigid in its axial direction along at least 60% of its length. In some embodiments, the sleeve is substantially rigid in its axial direction along at least 70% of its length. In some embodiments, the sleeve is substantially rigid in its axial direction along at least 80% of its length. In some embodiments, the sleeve is substantially rigid in its axial direction along at least 90% of its length. In some embodiments, the sleeve is substantially rigid in its axial direction along substantially its entire length.

In some embodiments, the sleeve is in the form of a rolled sheet of material. In some embodiments, at least one of the termini of the rolled sheet along the longitudinal axis thereof has a tapered geometry.

In some embodiments, the sleeve comprises a plurality of rods which impart stiffness along the axial direction of the sleeve. In some embodiments, the rods are connected at alternate ends by flexible connectors. In some embodiments, the rods are arranged generally parallel to one another and are spaced from each other by flexible segments.

In some embodiments, the sleeve is a radially expandable sleeve, and the method includes expanding the sleeve to the expanded state. In some embodiments, the sleeve is biased toward an expanded state. In some embodiments, the sleeve is biased toward an unexpanded state. In some embodiments, the sleeve comprises a shape memory material or a thermoplastic material which can be returned to an unexpanded state.

In some embodiments, the sleeve comprises one or more covering layers which are impermeable to mucous, saliva and other bodily fluids. In some embodiments, at least one covering layer is made of latex, polyurethane or butyl rubber.

In some embodiments, the sleeve comprises a deformable outer layer. In some embodiments, the deformable out layer is filled with a fluid. In some embodiments, the fluid is selected from air and a gel.

In some embodiments, the cuff is a balloon, the length of which is less than the length of the sleeve. In some embodiments, balloon is generally ring- or donut-shaped. In some embodiments, along at least an inner circumference of the balloon the balloon contacts the tube and along at least an outer circumference of the balloon the balloon contacts the inner surface of the sleeve. In some embodiments, along said at least an inner circumference the balloon is attached to said tube. In some embodiments, the balloon is attached to the tube by gluing or ultrasonic welding. In some embodiments, along said at least an inner circumference said balloon substantially sealingly contacts said tube. In some embodiments, along said at least an outer circumference the balloon is attached to said sleeve. In some embodiments, the balloon is attached to the sleeve by gluing or ultrasonic welding. In some embodiments, along said at least an outer circumference said balloon substantially sealingly contacts said sleeve. In some embodiments, the balloon is formed integrally with said tube. In some embodiments, the balloon is formed integrally with said sleeve.

In some embodiments, the balloon contacts the inner surface of said sleeve along not more than 50% of said inner surface. In some embodiments, the balloon contacts the inner surface of said sleeve along not more than 40% of said inner surface. In some embodiments, the balloon contacts the inner surface of said sleeve along not more than 30% of said inner surface. In some embodiments, the balloon contacts the inner surface of said sleeve along not more than 20% of said inner surface. In some embodiments, the balloon contacts the inner surface of said sleeve along not more than 10% of said inner surface.

In some embodiments, the length of said balloon along the longitudinal axis thereof is less than the length of said sleeve along the longitudinal axis thereof. In some embodiments, the length of said balloon along the longitudinal axis thereof is not more than half the length of said sleeve along the longitudinal axis thereof. In some embodiments, the length of said balloon along the longitudinal axis thereof is not more than 40% of the length of said sleeve along the longitudinal axis thereof. In some embodiments, the length of said balloon along the longitudinal axis thereof is not more than 30% of the length of said sleeve along the longitudinal axis thereof. In some embodiments, the length of said balloon along the longitudinal axis thereof is not more than 20% of the length of said sleeve along the longitudinal axis thereof. In some embodiments, the length of said balloon along the longitudinal axis thereof is not more than 10% of the length of said sleeve along the longitudinal axis thereof.

In some embodiments, the ratio of the length of said balloon along the longitudinal axis thereof to the length of said sleeve along the longitudinal axis thereof is less than 1:2. In some embodiments, the ratio of the length of said balloon along the longitudinal axis thereof to the length of said sleeve along the longitudinal axis thereof is not more than 1:5.

In some embodiments, the balloon is inflated. In some embodiments, the average pressure exerted by the outer surface of said sleeve against the wall of said trachea over a period of one minute does not exceed 25 mm Hg. In some embodiments, the pressure exerted by the outer surface of the sleeve against the wall of the trachea is less than 50 mg Hg for over 50% of the breathing cycle of the intubated patient. In some embodiments, the pressure of the cuff balloon is maintained fixed at pressure higher than 25 mm mercury independent of the human respiratory cycle during intubation. In some embodiments, the balloon is not inflated and an outer circumference of said balloon is sealingly adhered to the inner surface of said sleeve.

In some embodiments, the cuff is a flexible membrane. In some embodiments, the flexible membrane is generally disk-, ring- or cone-shaped. In some embodiments, along at least an inner circumference of the membrane the membrane contacts the tube and along at least an outer circumference of the membrane the membrane contacts the inner surface of the sleeve. In some embodiments, along said at least an inner circumference the membrane is attached to said tube. In some embodiments, the membrane is formed integrally with the tube. In some embodiments, the membrane is attached to the tube by gluing or ultrasonic welding. In some embodiments, along said at least an inner circumference said membrane substantially sealingly contacts said tube. In some embodiments, along said at least an outer circumference the membrane is attached to said sleeve. In some embodiments, the membrane is attached to the sleeve by gluing or ultrasonic welding. In some embodiments, along said at least an outer circumference said membrane substantially sealingly contacts said sleeve. In some embodiments, the membrane is formed integrally with said sleeve. In some embodiments, the membrane is adapted to allow movement of the tube along the longitudinal axis thereof without causing movement of the sleeve, while maintaining contact with the sleeve.

In some embodiments, the tracheal tube is an endotracheal tube. In some embodiments, the tracheal tube is a tracheostomy tube.

In some embodiments, a portion of the tube which is not in contact with the cuff can extend and/or contract along the longitudinal axis of the tube. In some embodiments, the portion is has an accordion-like or helical structure.

In some embodiments, the method further comprises inserting into the trachea an aeration structure for blowing air through the region of the trachea proximal to the cuff, the aeration structure having an outlet positioned on the proximal side of the cuff. In some embodiments, the aeration structure is a secondary lumen embedded in the wall of the tracheal tube.

In some embodiments, in comparison to intubation in ICU patients using conventional balloon cuff intubation, the method shows a decrease in the occurrence of at least one of the following: ventilation associated pneumonia; anoxia; erosion of the mucosa; destruction of tracheal cartilage rings; segmental tracheomalacia with dilatation of the trachea; full thickness erosion, optionally with perforation of the inominate artery anteriorly or posteriorly into the esophagus; and late complications of tracheal stenosis.

There is also provided, in accordance with an embodiment of the invention, a tracheal tube system, comprising: a tracheal tube having a distal end which is insertable into a trachea and a proximal end; a sleeve through which said tracheal tube passes, said sleeve having an inner surface which faces said tracheal tube and an outer surface and proximal and distal ends which correspond to the proximal and distal ends, respectively, of said tracheal tube; and a cuff which contacts at least a portion of the outer surface of said tracheal tube and at least a portion of said inner surface of said sleeve in a manner which substantially creates a seal between the distal and proximal ends of the inner surface of said sleeve and the distal and proximal ends of the outer surface of said tracheal tube.

There is also provided, in accordance with an embodiment of the invention, a system comprising: (a) a tracheal tube; (b) a sleeve which is sized to fit in a human trachea in the region between the larynx and the carina, the inner diameter of the sleeve being larger than the outer diameter of the tracheal tube; and (c) a cuff which traverses the region between the tracheal tube and the sleeve; the tracheal tube, the sleeve and the cuff being configured such than when deployed in a human trachea, the cuff, in combination with the tracheal tube and the sleeve, substantially prevents leakage to the lungs of fluids from the region of the trachea proximal to the larynx.

In some embodiments, the sleeve is sized and shaped to contact the tracheal wall. In some embodiments, the sleeve has a substantially circular cross-section. In some embodiments, the maximum diameter of the sleeve is not more than about 2.5 cm. In some embodiments, the sleeve has a non-circular cross section. In some embodiments, the sleeve is sized and shaped to contact the tracheal wall in a manner which substantially seals the lungs from the pharynx. In some embodiments, the sleeve is of a length that fits between the larynx and the carina. In some embodiments, the sleeve has a length of from 2 to 8 cm. In some embodiments, the sleeve is located in a human trachea and the distal end thereof is positioned 2 to 6 cm above the carina. In some embodiments, the average radius between the outer surface of the tube and inner surface of the sleeve is at least 1 mm.

In some embodiments, the tube is of substantially circular cross section and inner diameter of the tube is from 6 to 14 mm In some embodiments, the inner diameter of the tube is at least 6 mm. In some embodiments, the inner diameter of the tube is at least 7 mm. In some embodiments, the inner diameter of the tube is at least 8 mm. In some embodiments, the inner diameter of the tube is at least 9 mm. In some embodiments, the inner diameter of the tube is at least 10 mm. In some embodiments, the inner diameter of the tube is at least 11 mm In some embodiments, the inner diameter of the tube is at least 12 mm. In some embodiments, the inner diameter of the tube is at least 13 mm. In some embodiments, the inner diameter of the tube is 14 mm.

In some embodiments, the tube is of substantially circular cross section and outer diameter of the tube is from 8 to 16 mm. In some embodiments, the outer diameter of the tube is at least 8 mm. In some embodiments, the outer diameter of the tube is at least 9 mm. In some embodiments, the outer diameter of the tube is at least 10 mm. In some embodiments, the outer diameter of the tube is at least 11 mm. In some embodiments, the outer diameter of the tube is at least 12 mm. In some embodiments, the outer diameter of the tube is at least 13 mm. In some embodiments, the outer diameter of the tube is at least 14 mm. In some embodiments, the outer diameter of the tube is at least 15 mm. In some embodiments, the outer diameter of the tube is 16 mm.

In some embodiments, the ratio of the average outer radius of the sleeve to the outer radius of the tube is from 25:16 to 25:8. In some embodiments, the ratio of the average outer radius of the sleeve to the outer radius of the tube is from 25:15 to 25:8. In some embodiments, the ratio of the average outer radius of the sleeve to the outer radius of the tube is from 25:14 to 25:8. In some embodiments, the ratio of the average outer radius of the sleeve to the outer radius of the tube is from 25:13 to 25:8. In some embodiments, the ratio of the average outer radius of the sleeve to the outer radius of the tube is from 25:12 to 25:8. In some embodiments, the ratio of the average outer radius of the sleeve to the outer radius of the tube is from 25:11 to 25:8. In some embodiments, the ratio of the average outer radius of the sleeve to the outer radius of the tube is from 25:10 to 25:8. In some embodiments, the ratio of the average outer radius of the sleeve to the outer radius of the tube is from 25:9 to 25:8. In some embodiments, the ratio of the average outer radius of the sleeve to the outer radius of the tube is from 25:16 to 25:9. In some embodiments, the ratio of the average outer radius of the sleeve to the outer radius of the tube is from 25:16 to 25:10. In some embodiments, the ratio of the average outer radius of the sleeve to the outer radius of the tube is from 25:16 to 25:11. In some embodiments, the ratio of the average outer radius of the sleeve to the outer radius of the tube is from 25:16 to 25:12. In some embodiments, the ratio of the average outer radius of the sleeve to the outer radius of the tube is from 25:16 to 25:13. In some embodiments, the ratio of the average outer radius of the sleeve to the outer radius of the tube is from 25:16 to 25:14. In some embodiments, the ratio of the average outer radius of the sleeve to the outer radius of the tube is from 25:16 to 25:15.

In some embodiments, the sleeve is substantially rigid in its axial direction along at least 50% of its length. In some embodiments, the sleeve is substantially rigid in its axial direction along at least 60% of its length. In some embodiments, the sleeve is substantially rigid in its axial direction along at least 70% of its length. In some embodiments, the sleeve is substantially rigid in its axial direction along at least 80% of its length. In some embodiments, the sleeve is substantially rigid in its axial direction along at least 90% of its length. In some embodiments, the sleeve is substantially rigid in its axial direction along substantially all of its length.

In some embodiments, the sleeve is in the form of a rolled sheet. In some embodiments, at least one of the termini of the sheet along the longitudinal axis thereof has a tapered geometry.

In some embodiments, the sleeve comprises a plurality of rods which impart stiffness along the axial direction of the sleeve. In some embodiments, the rods are connected at alternate ends by flexible connectors. In some embodiments, the rods are arranged generally parallel to one another and are spaced from each other by flexible segments.

In some embodiments, the sleeve is a radially expandable sleeve which is in the expanded state. In some embodiments, the sleeve is biased toward an expanded state. In some embodiments, the sleeve is biased toward an unexpanded state. In some embodiments, the sleeve comprises a shape memory material or a thermoplastic material which can be returned to an unexpanded state.

In some embodiments, the sleeve comprises one or more covering layers which are impermeable to mucous, saliva and other bodily fluids. In some embodiments, at least one covering layer is made of latex, polyurethane or butyl rubber.

In some embodiments, the sleeve comprises a deformable outer layer. In some embodiments, the deformable out layer is filled with a fluid. In some embodiments, the fluid is selected from air and a gel.

In some embodiments, the cuff is a balloon, the length of which is less than the length of said sleeve. In some embodiments, the balloon is generally ring- or donut-shaped. In some embodiments, along at least an inner circumference of the balloon the balloon contacts the tube and along at least an outer circumference of the balloon the balloon contacts the inner surface of the sleeve. In some embodiments, along the at least an inner circumference the balloon is attached to said tube. In some embodiments, the balloon is attached to the tube by gluing or ultrasonic welding. In some embodiments, along said at least an inner circumference said balloon substantially sealingly contacts said tube. In some embodiments, along said at least an outer circumference the balloon is attached to said sleeve. In some embodiments, the balloon is attached to the sleeve by gluing or ultrasonic welding. In some embodiments, along said at least an outer circumference said balloon substantially sealingly contacts said sleeve.

In some embodiments, the balloon contacts the inner surface of said sleeve along not more than 50% of said inner surface. In some embodiments, the balloon contacts the inner surface of said sleeve along not more than 40% of said inner surface. In some embodiments, the balloon contacts the inner surface of said sleeve along not more than 30% of said inner surface. In some embodiments, the balloon contacts the inner surface of said sleeve along not more than 20% of said inner surface. In some embodiments, the balloon contacts the inner surface of said sleeve along not more than 10% of said inner surface.

In some embodiments, the length of said balloon along the longitudinal axis thereof is less than the length of said sleeve along the longitudinal axis thereof. In some embodiments, the length of said balloon along the longitudinal axis thereof is not more than half the length of said sleeve along the longitudinal axis thereof. In some embodiments, the length of said balloon along the longitudinal axis thereof is not more than 40% of the length of said sleeve along the longitudinal axis thereof. In some embodiments, the length of said balloon along the longitudinal axis thereof is not more than 30% of the length of said sleeve along the longitudinal axis thereof. In some embodiments, the length of said balloon along the longitudinal axis thereof is not more than 20% of the length of said sleeve along the longitudinal axis thereof. In some embodiments, the length of said balloon along the longitudinal axis thereof is not more than 10% of the length of said sleeve along the longitudinal axis thereof.

In some embodiments, the ratio of the length of said balloon along the longitudinal axis thereof to the length of said sleeve along the longitudinal axis thereof is less than 1:2. In some embodiments, the ratio of the length of said balloon along the longitudinal axis thereof to the length of said sleeve along the longitudinal axis thereof is not more than 1:5.

In some embodiments, the balloon is inflated. In some embodiments, the system is located in a trachea and the average pressure exerted by the outer surface of said sleeve against the wall of said trachea over a period of one minute does not exceed 25 mm Hg. In some embodiments, the system is located in a trachea and the pressure exerted by the outer surface of the sleeve against the wall of the trachea is less than 50 mg Hg for over 50% of the breathing cycle of the intubated patient. In some embodiments, system is located in a trachea and the pressure of the cuff balloon is maintained fixed at pressure higher than 25 mm mercury independent of the human respiratory cycle during intubation. In some embodiments, the balloon substantially immobilizes the tube relative to the sleeve in the longitudinal direction thereof. In some embodiments, the balloon is not inflated and an outer circumference of said balloon is sealingly adhered to the inner surface of said sleeve.

In some embodiments, the cuff is a flexible membrane. In some embodiments, the flexible membrane is generally disk-, ring- or cone-shaped. In some embodiments, along at least an inner circumference of the membrane the membrane contacts the tube and along at least an outer circumference of the membrane the membrane contacts the inner surface of the sleeve. In some embodiments, along said at least an inner circumference the membrane is attached to said tube. In some embodiments, the membrane is attached to the tube by gluing or ultrasonic welding. In some embodiments, the membrane is formed integrally with the tube. In some embodiments, along said at least an inner circumference said membrane substantially sealingly contacts said tube. In some embodiments, along said at least an outer circumference the membrane is attached to said sleeve. In some embodiments, the membrane is formed integrally with the sleeve. In some embodiments, the membrane is attached to the sleeve by gluing or ultrasonic welding. In some embodiments, along said at least an outer circumference said membrane substantially sealingly contacts said sleeve. In some embodiments, the membrane is adapted to allow movement of the tube along the longitudinal axis thereof without causing movement of the sleeve, while maintaining contact with the sleeve.

In some embodiments, a portion of the tube which is not in contact with the mediating structure can extend and/or contract along the longitudinal axis of the tube.

In some embodiments, the system further comprises an aeration structure having a distal end which opens near the cuff on the proximal side of the cuff and a proximal end that opens near the proximal end of the tube. In some embodiments, the aeration structure is a secondary lumen embedded in the wall of the tracheal tube.

In some embodiments, the tracheal tube is an endotracheal tube. In some embodiments, the tracheal tube is a tracheostomy tube.

There is also provided, in accordance with an embodiment of the invention, a tracheal tube having an inflatable cuff, wherein a portion of the tube which is not in contact with the cuff can extend and/or contract along the longitudinal axis of the tube. In some embodiments, the portion is has an accordion-like or helical structure.

There is also provided, in accordance with an embodiment of the invention, a tracheal intubation kit, comprising: (a) at least one of (i) a tracheal tube having a distal end which is insertable into a trachea and a proximal end; (ii) a sleeve through which said tracheal tube can pass, said sleeve having an inner surface and an outer surface and proximal and distal ends which correspond to the proximal and distal ends, respectively, of said tracheal tube; and (iii) a cuff which is positionable between the tube and sleeve to contact at least a portion of the outer surface of said tracheal tube and at least a portion of said inner surface of said sleeve in a manner which substantially creates a seal between the distal and proximal ends of the inner surface of said sleeve and the distal and proximal ends of the outer surface of said endotracheal or tracheostomy tube; and (b) instructions which instruct a user to how to align, in the trachea of a patient, said tracheal tube, said sleeve, and said cuff, such that said cuff contacts at least a portion of the outer surface of said tracheal tube and at least a portion of said inner surface of said sleeve in a manner which substantially creates a seal between the distal and proximal ends of the inner surface of said sleeve and the distal and proximal ends of the outer surface of said tracheal tube, whereby to radially space the tube from the sleeve.

There is also provided, in accordance with an embodiment of the invention, a tracheal intubation kit comprising a system as described herein and instructions instructing a user how to deploy said system in the trachea of a human patient.

There is also provided, in accordance with an embodiment of the invention, a tracheal intubation kit comprising (a) at least one of (i) a tracheal tube having a distal end which is insertable into a trachea and a proximal end; (ii) a sleeve through which said tracheal tube can pass, said sleeve having an inner surface and an outer surface and proximal and distal ends which correspond to the proximal and distal ends, respectively, of said tracheal tube; and (iii) a cuff which is positionable between the tube and sleeve to contact at least a portion of the outer surface of said tracheal tube and at least a portion of said inner surface of said sleeve in a manner which substantially creates a seal between the distal and proximal ends of the inner surface of said sleeve and the distal and proximal ends of the outer surface of said endotracheal or tracheostomy tube; and (b) instructions which instruct a user how to use the kit to practice a method as described herein.

### BRIEF DESCRIPTION OF THE DRAWINGS

Embodiments of the invention will be explained below in greater detail with reference to the accompanying illustrative and non-limitative drawings, in which:

FIG. 1 illustrates a typical design and employment of an endotracheal tube (ETT), as known in the art (in this case taken from FIG. 2A of U.S. Pat. No. 6,843,250);

FIG. 2 presents drawings showing schematically elements of an endotracheal tube with sleeve, in accordance with some embodiments of the invention;

FIG. 3 diagrams forces operating at the contact between the balloon cuff and the sleeve support;

FIG. 4 illustrates the characteristic pooling of fluids both as known in the art and in accordance with some embodiments of the present invention;

FIG. 5 illustrates an embodiment of suction elements on the sleeve;

FIG. 6 illustrates a prior art ETT balloon with a unique cup shape (taken from U.S. Pat. No. 4,979,505);

FIG. 7 illustrates the difference in the influence of air pressure on the sealing of intubation elements against the trachea wall in an embodiment of the present invention in comparison to the prior art;

FIG. 8 illustrates additional embodiments of the present invention;

FIG. 9 illustrates a sleeve structure in accordance with some embodiments of the invention;

FIG. 10 shows force vectors contributions of the bare sleeve pressure and the cuff balloon pressure;

FIG. 11 illustrates some embodiments of the sleeve structure;

FIG. 12 illustrates another embodiment useable in the sleeve structure.

FIG. 13 illustrates a spiral folded sleeve in accordance with embodiments of the present invention;

FIG. 14 illustrates a cross section of a sleeve in accordance with some embodiments of the invention;

FIG. 15 illustrates an embodiment of the invention where the sleeve is connected to the ETT tube via a flexible membrane;

FIG. 16 illustrates an embodiment of the invention having a springy mid-section of the endotracheal tube below the level of the vocal cords;

FIG. 17 illustrates an embodiment of the invention having a springy mid-section of the endotracheal tube above the level of the vocal cords;

FIG. 18 illustrates an embodiment of the invention having air outlets opening from a second lumen at a position below the level vocal cords; and

FIG. 19 illustrates a system in accordance with some embodiments of the invention.

### DETAILED DESCRIPTION

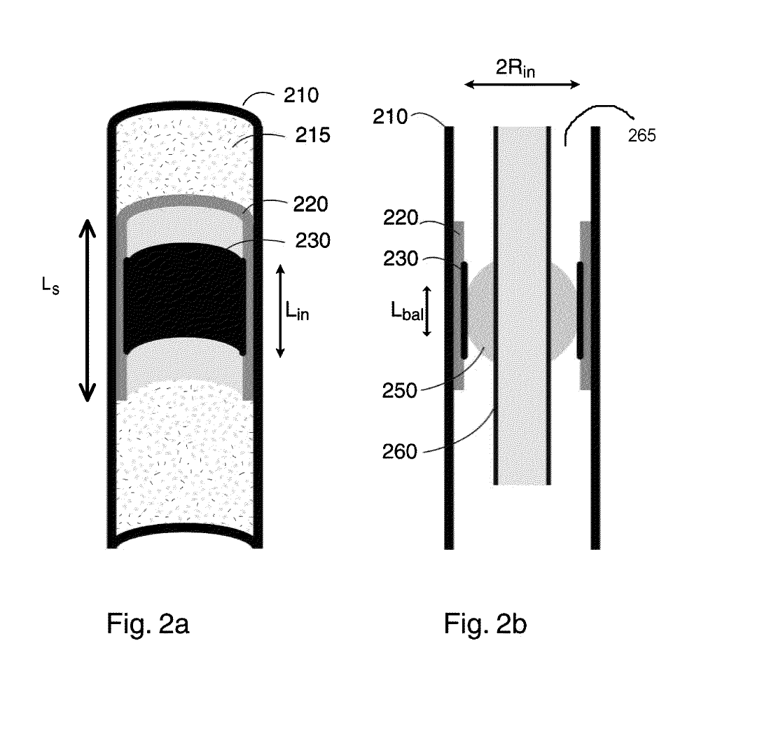

In accordance with embodiments of the invention, when the ETT is deployed, it is deployed in a such as way that a portion of the ETT contacts a “sleeve” element via a mediating element, thus creating a seal within the sleeve and between the lungs and the oral cavity. FIGS. 2a and 2b show in cut-away perspective and cross-sectional view along the longitudinal axis, respectively, an example of such a sleeve element 220, constructed and operative in accordance with embodiments of the present invention, deployed in a trachea 210. As shown, sleeve element 220 is of quasi-cylindrical shape, and of length Lₛ. It will also be appreciated that for purposes of the present discussion, sleeve element 220 is an expandable sleeve element in an expanded state; such expandable sleeve elements will be discussed in greater detail below.

As shown in both FIGS. 2a and 2b, the outer surface of sleeve element 220 is in contact with the tracheal tissue; as shown in FIG. 2b, which in addition to sleeve element 220 also shows an endotracheal tube 260 having a balloon cuff 250, a sealing contact is established between a portion of the inner surface of sleeve 220 and balloon cuff 250, which is attached to and inflated around ETT 260. Thus, as will be explained in greater detail below, when the pressure exerted by sleeve 220 on trachea 210 (which may itself arise from pressure exerted by balloon cuff 250 on sleeve element 220) is sufficient, in the region 265 between the trachea and the ETT 260 (hereinafter referred to as the “interstitial region”), a seal is created between the lungs on the distal end of sleeve element 220 and the oral cavity on the proximal end of sleeve element 220, thus ensuring that air can be forced into and withdrawn from the lungs through the ETT, as air will be unable to pass into or out of the lungs through the interstitial region. Furthermore, to the extent that fluids enter the trachea, the seal between sleeve element 220 and ETT 260 confines the collected fluids to the region within the sleeve, away from contact with the tracheal wall tissue. For ease of reference, the use of a sleeve together with an ETT may in some places be referred to as “sleeve-supported ETT”.

The term “sleeve” or “sleeve element” is used so as to intuitively call to mind the image of a generally cylindrical shaped element which can be expanded to press snugly against the inner wall of biological tubes, such as the trachea. A sleeve element may thus be reminiscent of stent devices, but this mental association is not meant to limit the sleeve elements discussed herein to the shapes or designs or constructions of stent devices presently known in the art or to imply that currently known stent devices are necessarily usable in accordance with embodiments of the present invention. Further properties of sleeve elements in accordance with embodiments of the present invention will be discussed further below.

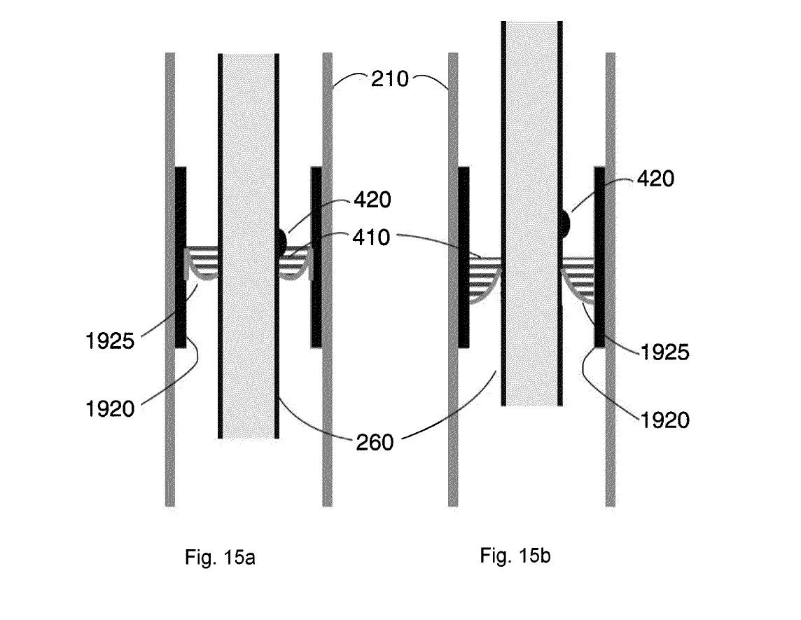

Although as shown in FIG. 2b, the sealing between the ETT and the sleeve element is effected by a cuff balloon, such sealing can be effected in other ways as well, for example by physical attachment of the mediating element to the outer surface of the ETT on the one hand and to a portion of the inner surface of the sleeve on the other hand, e.g. a deformable membrane such as an elastic diaphragm (as will be discussed in more detail with respect to FIG. 15), or via an iris closing from the sleeve.

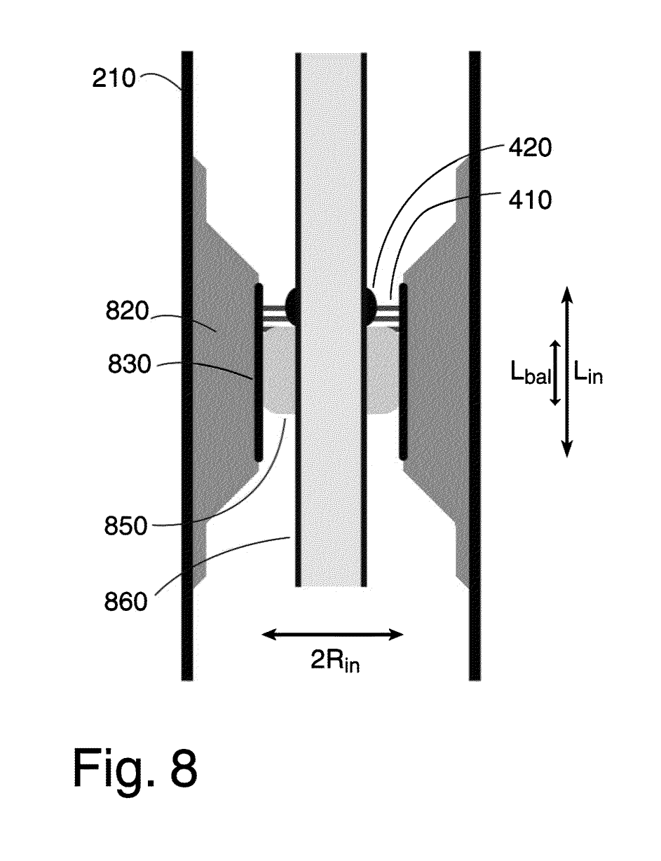

It will be appreciated that a in FIG. 2b, part of the inner surface of sleeve element 220 has a ring-like region 230 of radius Rᵢₙ (diameter 2Rᵢₙ) and rigid support along length Lᵢₙ, wherein Lᵢₙ≦Lₛ. (Although as shown in FIG. 2a, 230 protrudes outwardly from the inner surface of sleeve element 220 this need not be the case, and 230 may be flush with the inner surface of sleeve element 220.) As will be explained below, the longitudinal rigidity along the length Lᵢₙ enables sleeve element 220 to disperse the pressure applied by balloon cuff 250 over a wider area, thus mitigating the effective pressure applied to the trachea; but radial flexibility along radius Rᵢₙ, up to a predetermined maximum radius Rᵢₙ₋ₘₐₓ, is allowable. It will be appreciated that although the cuff balloon 250 shown in FIG. 2b, appears to be of a generally spherical shape with a hole in the middle through which tube 260 passes and contacts cuff balloon 250, i.e. it is in the shape of torus in which the inner edge of the torus has been extended up and down in the axial direction, in principle cuff balloon 250 could have any other suitable shape, such as a donut or ring shape, including an oblong donut shape such as is shown for cuff balloon 850 in FIG. 8. Irrespective of the shape of cuff balloon 250, the ETT cuff balloon, when inflated, has a contact length L_{bal} with the sleeve support; in general, Lᵢₙ>L_{bal}.

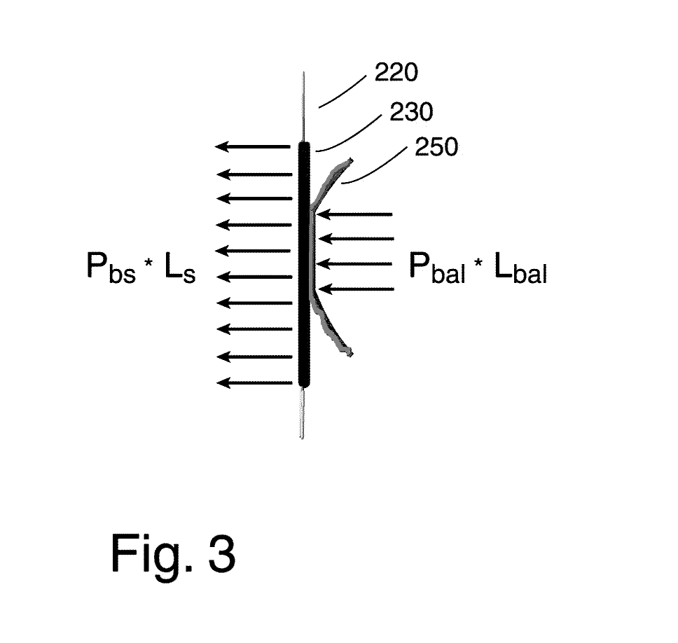

A novel property of construction shown in FIG. 2b is a mediating degree of freedom between the pressure exerted by the balloon cuff and the pressure exerted on the trachea wall tissue is obtained. This will be better understood with reference to FIG. 3, which schematically shows how pressure P_{bal} of balloon cuff 250 is mediated to lower pressure P_{bs} across a region 230 of sleeve element 220 to the trachea wall, where the transmitted pressure P_{bs}=(L_{bal}/Lᵢₙ) P_{bal}. The length Lᵢₙ is assumed to be substantially rigid, corresponding generally to the length Lₛ of the sleeve, but the length of the rigid portion, Lᵢₙ, may be less than the length of the entire sleeve, Lₛ.

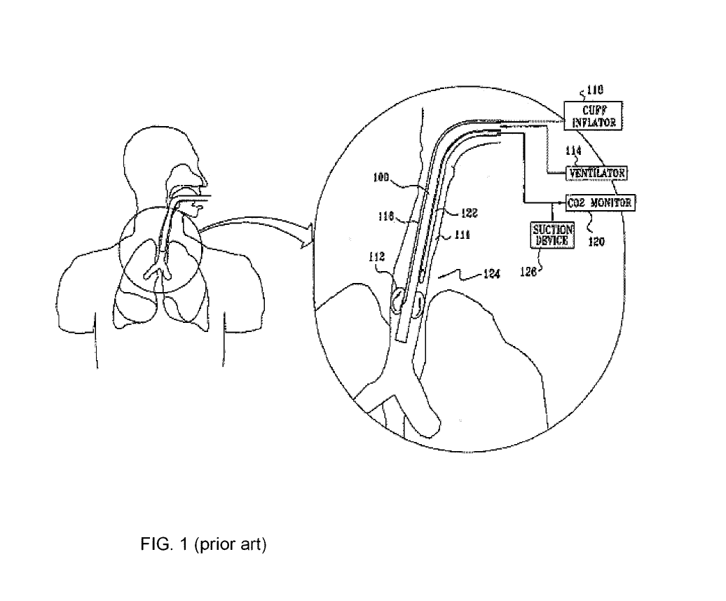

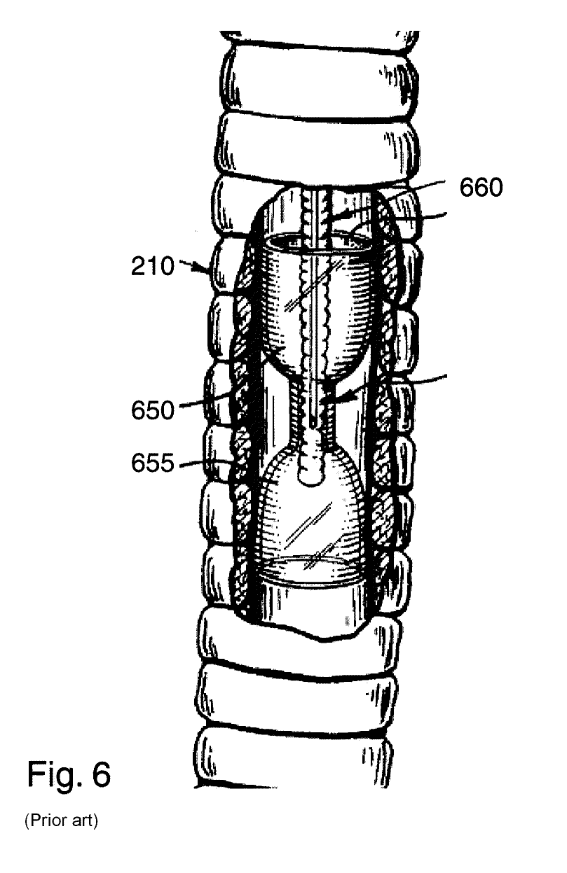

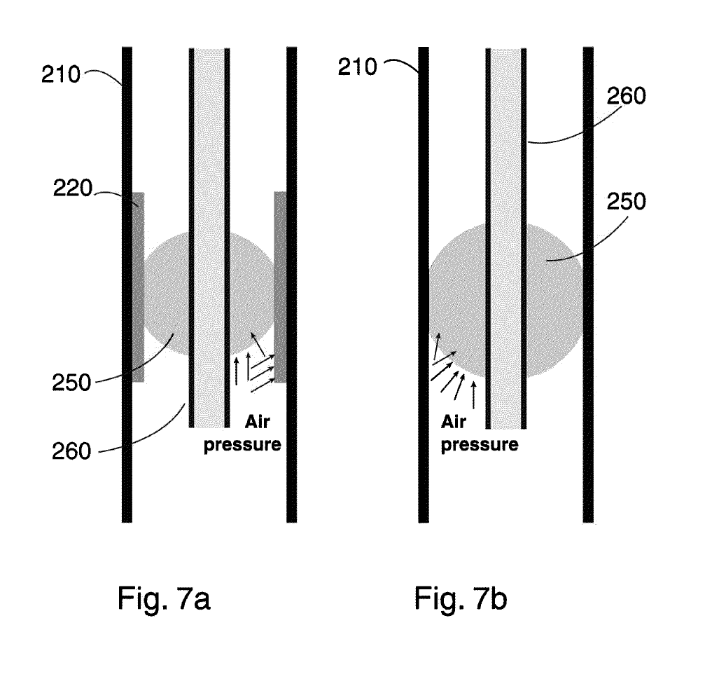

As shown in FIG. 7a, emplacement of the balloon cuff 250 within a sleeve 220 also ensures that the seal between the sleeve and the trachea increases during inspiration, as air pressure on the distal side of the cuff (the nearer the lungs) presses against both the balloon cuff and the sleeve. As airway pressure fluctuates with ventilatory requirements of the lungs, this pressure is transmitted to the inner surface of the sleeve and increases the seal between the sleeve and the tracheal wall. The higher the pressure within the tracheobronchial system, the tighter the seal, with maximum pressure occurring at the end of the inspiratory phase of respiration. In this sense, the principles at work are similar to those outlined in U.S. Pat. No. 4,979,505, illustrated in present FIG. 6 (which is taken from FIG. 1 of the '505 patent). Thus, there is intermittent compression of the capillaries only for the duration of high pressure phases of respiration, thus avoiding tissue necrosis associated with issue anoxia that accompanies the prolonged use of high cuff pressure.

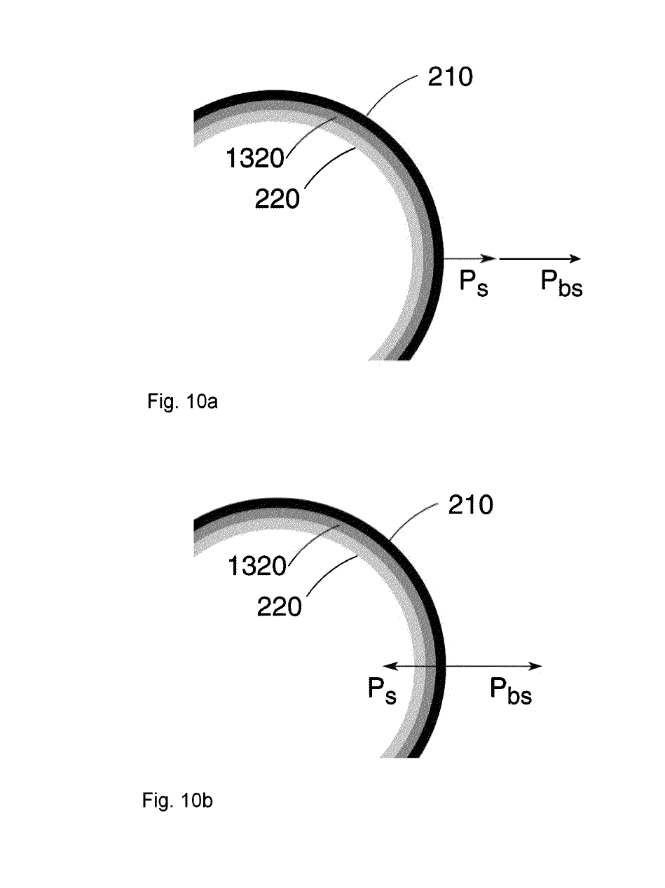

As illustrated in FIGS. 10a and 10b, which show cross-sections of an expandable sleeve element taken perpendicular to the longitudinal axis, in addition to mediating a lower pressure from the cuff balloon to the trachea, an additional degree of freedom may be provided in the form of the inherent tension in sleeve element 220. This tension may be chosen to affect the total pressure Pₜ mediated to the trachea. Thus, in one embodiment, illustrated schematically in FIG. 10a, the bare sleeve has an expansion tension to press against the trachea wall, so that, on its own, the sleeve exerts a pressure Pₛ on the trachea. Therefore, when the balloon is inflated to pressure P_{bal}, the total pressure exerted on the trachea wall is Pₜ=P_{bs}+Pₛ. Pₜ can still be substantially lower than P_{bal}.

In an alternative embodiment, illustrated schematically in FIG. 10b, the bare sleeve has a compression tension to contract away from the trachea wall. Thus, on its own the sleeve exerts a compression force Pₛ on the inflated balloon cuff. Therefore, when the balloon cuff is inflated to pressure P_{bal}, expanding the sleeve to press against the trachea wall, the total pressure exerted on the trachea wall is Pₜ=P_{bs}−Pₛ. In such an embodiment, Pₜ is inherently lower than P_{bal}.

It will be appreciated that in the embodiment shown in FIG. 10b, sleeve re-collapse and removal after use is readily facilitated. The situation of FIG. 10b can be realized when the sleeve is delivered in a non-compressed, weakly compressed, or even slightly stretched state (with respect to a free independent sleeve rest configuration). Then the sleeve is elastically expanded in diameter under the pressure force of the inflating ETT cuff balloon. When the balloon is deflated, the contracting elastic forces of the sleeve cause the sleeve to recollapse away from the trachea wall, without need for special additional re-collapse tools or force elements.

It will also be appreciated that, in principle, the radius of expansion of the sleeve may be limited, so that the inner radius of the sleeve are limited to a maximum radius Rᵢₙ₋ₘₐₓ. This can provide a protective decoupling of the balloon pressure from the sleeve pressure on the trachea beyond the maximum inner surface radius of the sleeve. The balloon can be inflated to its desired high pressure, while the sleeve, if separately inflatable (see description below), can be independently inflated to a different pressure.

It will be appreciated that any construction of the sleeve element that facilitates radial expansion thereof may be realized in alternative embodiments of the invention that take advantage of the controlled difference and relative independence between the sealing pressure P_{bal} applied within the cuff balloon of the ETT tube and the associated pressure P_{bs} exerted by the sleeve element on the trachea tissue. This enables the total pressure Pₜ of the sleeve element on the trachea wall tissue to be maintained below 25 mm mercury at most times during intubation, and thus tissue anoxia can be avoided even for extended intubation periods.

It will also be appreciated that the pressure decoupling between the ETT cuff balloon and the trachea tissue means that it is unnecessary to dynamically regulate the balloon pressure, so that the balloon pressure can be set to provide an adequate level of sealing, to prevent leak of fluids past the cuff (seeping through the contact between the cuff balloon and the sleeve). Hence, various costs and complications associated with dynamic trachea pressure-sensing and cuff pressure regulation of some prior art devices can be eliminated.

With regard to the sealing of the contact between the cuff balloon and the sleeve, it will also be appreciated that since the shape, size, and material composition of the sleeve, including its inner radius, can be predetermined, an ETT balloon of well-fitting properties can be designed, which in some embodiments will obviate the need, found in some balloon cuffs presently in use, to inflate the cuff to a range of sizes in an attempt to fit the varying and imprecise shape of the human trachea wall, which can result in folds through which fluids can leak. Thus, for example, in accordance with some embodiments of the present invention, high-pressure, low-volume (HPLV) cuff balloons may be used instead of the presently preferred low-pressure, high-volume (LPHV) balloons. It will also be appreciated that in some embodiments, the inner surface of the sleeve may be roughened (e.g., by circular ribs) so that fluids are further obstructed from easily flowing into the lungs.

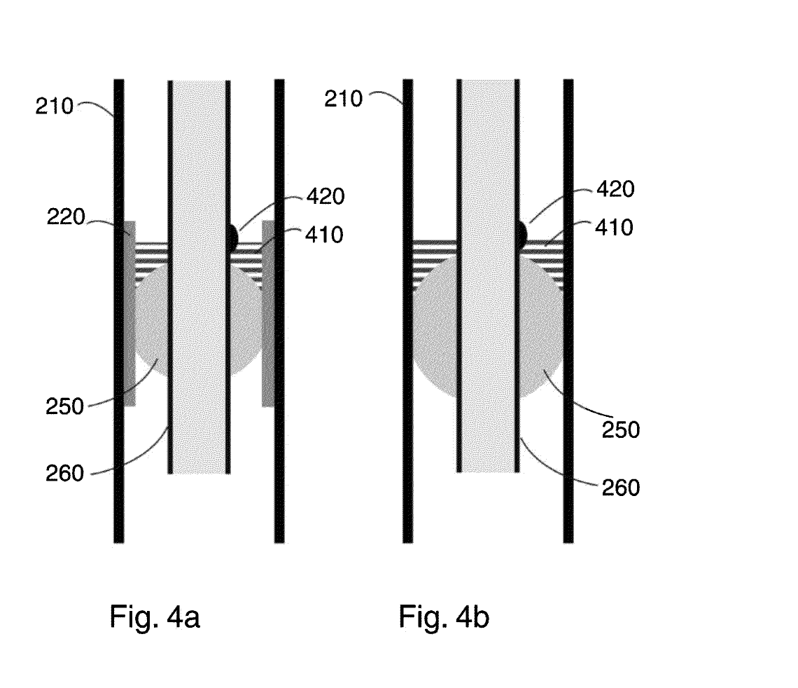

Attention is now drawn to FIGS. 4a and 4b. As illustrated in FIG. 4a, use of a sleeve element 220 in accordance with embodiments of the invention enables fluids 410 to pool and collect away from the trachea wall tissue 210; this is in sharp contrast to the known art of ETT tubes, in which, as illustrated in FIG. 4b, curvature of the inflated cuff 250 tends to cause fluids 410 to collect exactly at the highest risk location, viz. near the trachea skin tissue 210. As shown in FIG. 4a, in accordance with embodiments of the present invention, the fluids collect at the bottom of a toroidal-like space, the sides of which are formed by the walls of the sleeve 220 and the ETT 260, and a sealing bottom is formed by the inflated cuff balloon 250. Consequently, even if there are momentary breaks in the seal between the trachea tissue 210 and the sleeve 220, there are no significant volumes of fluids present at the interface between sleeve 220 and trachea 210 which can leak down into the lungs. Such an arrangement may also make the ETT/cuff/sleeve arrangement less sensitive to the occasional cough or other violent movement of the trachea than the ETT/cuff systems presently in use. Since the fluids are confined away from the trachea wall tissue, momentary breach of the sealed passage to the lungs does not necessarily result in leak of fluids into the lungs.

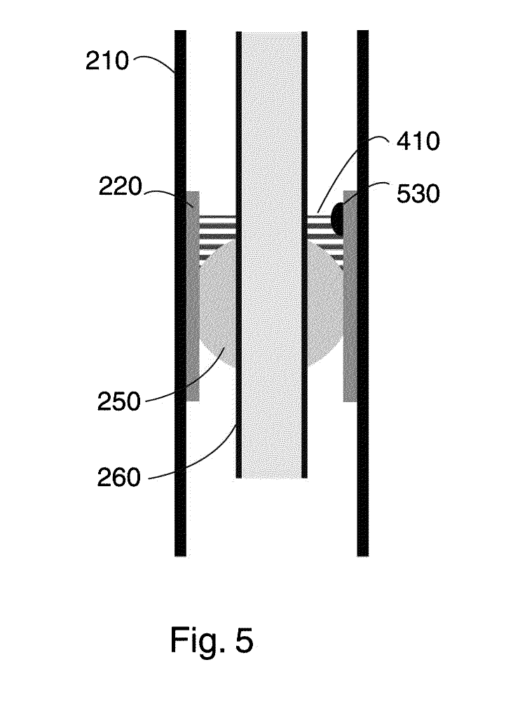

Moreover, as shown in FIG. 4b, as deployed when used in the prior art, suction elements 420 (which are connected to an external suction tube, not shown, that for example exits the patient's mouth) often cannot reach the contact location between the balloon 250 and the trachea tissue 210 where fluids 410 naturally collect due to the force of gravity, and where suction is most needed. In contrast, as shown in FIGS. 4a and 5, in embodiments of the present invention, the suction device elements 420 and 530, respectively, can be placed at locations on the ETT or the sleeve that enable them to collect a larger percentage of the fluids that collect than is the case with the types of devices illustrated in FIG. 4b.

In terms of construction and materials used for the balloon cuff, in accordance with embodiments of the present invention there the balloon cuff itself need not be excessively delicate in construction, since the balloon cuff is not in direct contact with the trachea tissue. Hence, if necessary suction elements can be brought into direct contact with and even touch the balloon cuff for better removal of accumulated fluids.

It will be appreciated that the foregoing discussion is predicated on the sleeve presenting, at least upon a substantial area thereof, an impermeable surface. This impermeable surface provides a barrier between the fluids collected on the inner side of the sleeve from coming in contact with the trachea tissue. Bare metal stents are permeable to fluids and are therefore unsuitable for use as a sleeve in accordance with embodiments of the present invention. Although polymer-coated stents are known (see, e.g., U.S. Pat. Nos. 7,300,459 and 7,374,570), and such stents can be made in a manner in which they are impermeable to fluids, such stents do not possess the necessary longitudinal rigidity for use in embodiments of the present invention such as depicted in FIG. 2.

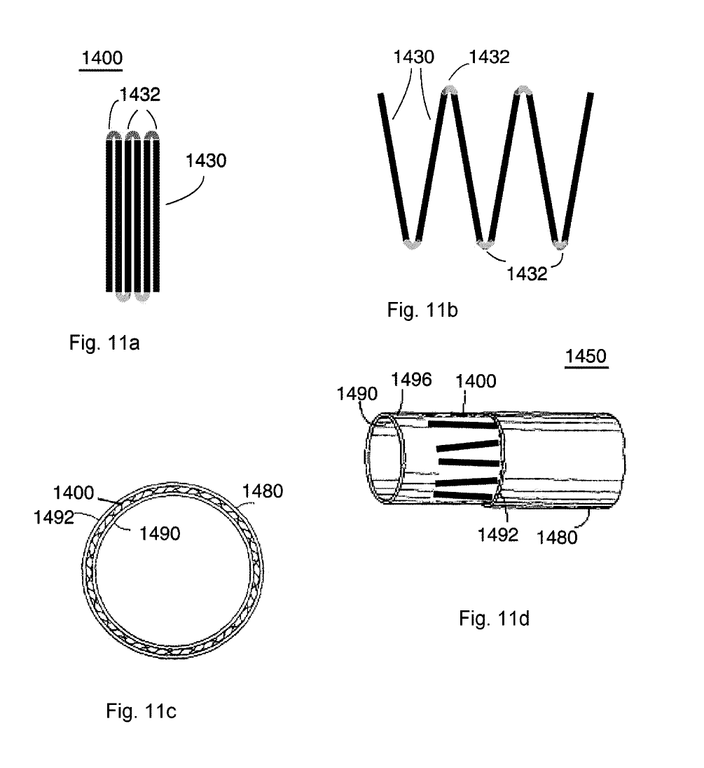

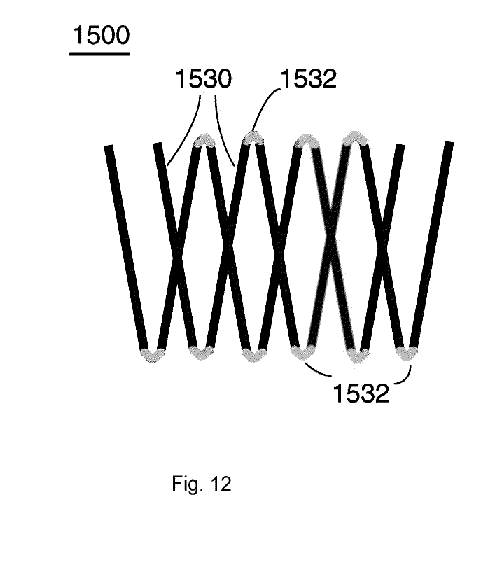

FIGS. 11 and 12 show embodiments in which a core skeleton, which provides longitudinal rigidity but allows for expansion radially, is covered by an impermeable sheet of malleable polymer material. FIGS. 11c and 11d show a sleeve structure 1450, which is made from a skeleton 1400 encased in outer polymer layer 1492 and an optional inner polymer layer 1490; it is outer surface 1480 that makes contact with the trachea. (FIG. 11c shows the sleeve of FIG. 11d in cross-sectional view, perpendicular to the longitudinal axis, approximately midway along the length of the sleeve.) At least one of the polymer layers is impermeable to the fluids that collect in the region formed by the ETT, the cuff balloon and sleeve. The sleeve skeleton 1400 is in the form of linked stiff rods 1430. The links 1432 (shown in FIGS. 11a and 11b but not FIG. 11c or 11d, which show only part of rods 1430) are flexible and alternate between top and bottom connection of neighboring rods. The stiffness of the rods 1430 provides sleeve 1400 with the rigidity along the longitudinal axis, while the links 1432 enable radial expansion and elastic return forces. Preferably, the length of each rod 1430 extends through more than 50% and more preferably through substantially the entire length of the sleeve. It will be appreciated that a break or elastic mid-section in the rods (akin to what is commonly found in stent constructions) would reduce the overall axial stiffness of the sleeve, resulting in a less even distribution of pressure along the sleeve on the trachea wall due to pressure exerted on the sleeve by a cuff balloon, and therefore rods 1430 are formed without such breaks or elastic mid-sections.

If in their relaxed state, links 1432 are in an open configuration, then a compressed sleeve held in place by a restraining force would self-expand upon release of the restraining force as the links return to their relaxed, open configuration. Conversely, if the free state of the links is relatively closely compact, then the sleeve will radially expand only under radial force (e.g., exerted by the cuff balloon) and will self contract upon relaxing of the radial force (e.g., upon deflation of the cuff balloon).

FIG. 12 shows a series of rods 1530, which are similar in construction to rods 1430, and which have flexible end pieces 1532 analogous to pieces 1432, and can be incorporated into a sleeve in like manner, but in FIG. 12 there are two sets of connected rods, providing additional rigidity along the longitudinal axis.

In accordance with some embodiments, core skeleton 1400, while encased between two polymer layers or covered with a single outer layer, is not glued or adhered to the polymer layer(s), and instead has some freedom of movement relative to the encasing layer(s), thus facilitating radial expansion. In those embodiments in which the skeleton is encased between two polymer layers or covered with a single outer layer, the polymers may be elastic, e.g. latex, to facilitate expansion of the sleeve.

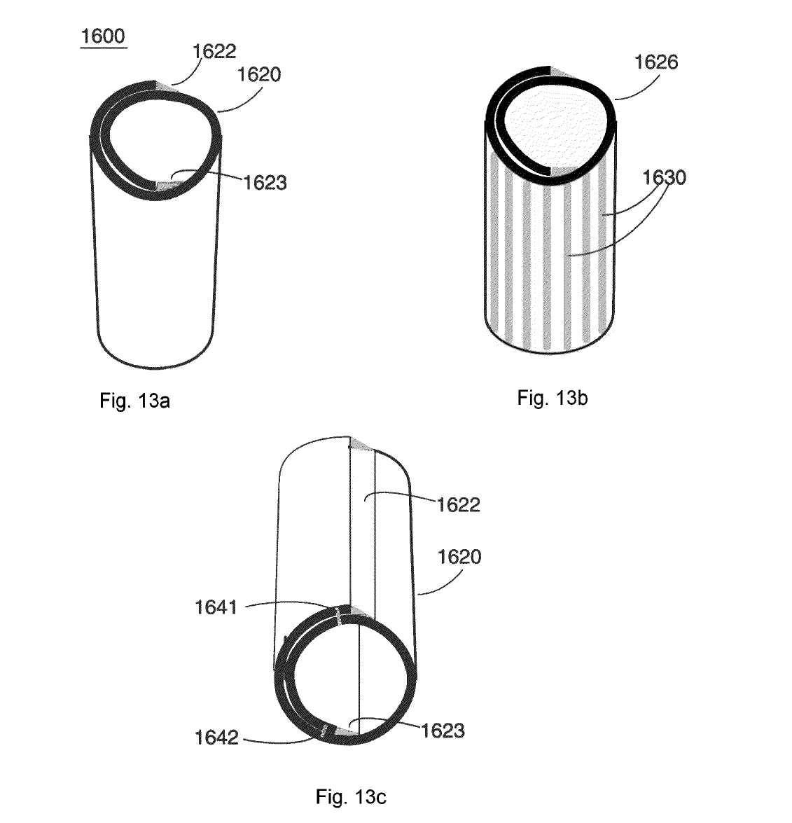

An alternative embodiment of the core skeleton element is in the form of a spiral or rolled sheet, which is free to expand under the coating envelope. Further discussion of embodiments of a rolled sheet are presented in the discussion of FIG. 13 below.

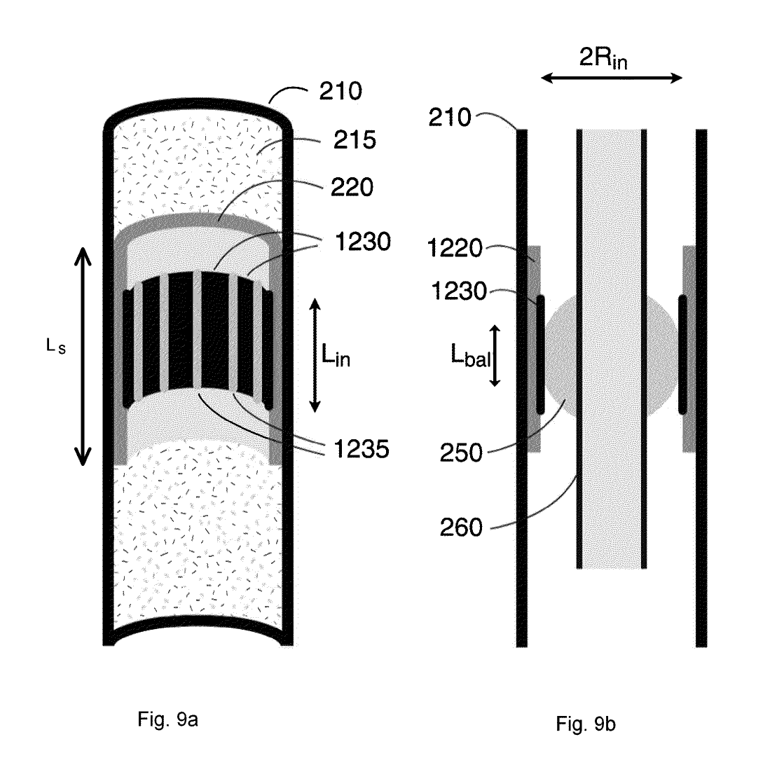

Another construction that can be used to simultaneously obtain longitudinal rigidity and radial flexibility (LRRF) in the sleeve is shown in FIGS. 9a and 9b, which correspond to FIGS. 2a and 2b. FIG. 9a shows that spaced intermittently along sleeve 220 are a plurality of rigid sections 1230 (which may be, for example, rod-like structures, or stiff plates), aligned with the longitudinal axis of the sleeve, and which are interspersed with elastic sections 1235. In some embodiments, the alternating rigid/elastic sections can be integrated into sleeve 220, which also touches trachea wall tissue on its outer side. In other embodiments, the alternating rigid/elastic sections can be inserted as an independent mediating element between the cuff balloon and a sleeve which is elastic in all directions. Thereby, as illustrated in FIG. 9b, the local cuff 250 expansion is mediated on the whole length Lᵢₙ of the LRRF element 1230 to the outer sleeve element 1220.

Reference is now made to FIG. 13, which illustrates several spiral rolled sleeves in accordance with embodiments of the present invention. As shown in FIG. 13a, spiral sleeve structure 1600 has the general form of a sheet 1620 (the sleeve “wall”) rolled into a spiral. Various alternative embodiments of the sheet construction can be realized; examples of such constructions are further elaborated below, but these are not meant to be limiting.

In the configuration shown for spiral sleeve 1600, the sleeve wall 1620 can optionally be constructed entirely or mostly from a sheet of non-stretchable material. Such a spiral sleeve can radially expand by unfolding of the spiral. Thus, a spiral sleeve configuration enables the sleeve to provide both a substantially stiff inner surface, i.e. longitudinal rigidity in the direction of the axis of coiling of the sleeve, and flexible radial expansion perpendicular to that axis, to allow the expanded sleeve to conform to the mostly oval but not perfectly cylindrical geometry commonly found in the trachea. The structure of the sleeve wall 1620 can be uniform both along the length and the width of the wall (i.e. both along the axis of coiling and in the sleeve body in the direction of the coiling), e.g. in some embodiments, the inner surface of the sleeve wall 1620 can be made of a non-stretching polymer or a metallic material.

In other embodiments, as shown in FIG. 13b, the structure of the sleeve wall 1620 can be non-uniform in the direction of the coiling of the sleeve, e.g., in some embodiments, the inner wall of the sleeve wall 1620 can be made of alternating rod-like sections of stiff polymer or metallic material 1630, interspersed by more pliable or elastic regions.

In yet other embodiments, the sleeve wall can take the form of a core skeleton (e.g., as illustrated in FIGS. 11 and 12) covered by an impermeable thin layer.

Typical spiral sleeve designs have imperfect sealing at both terminii of the spiral sheet parallel to the axis of coiling (see e.g. ends 1622 and 1623 in FIG. 13c), and thus, there will be risk of fluids leaking along these ends when such a sleeve is used in combination with a balloon-cuffed ETT in accordance embodiments of the present invention. To reduce the likelihood of this happening, as illustrated in FIGS. 13a, 13b and 13c, in some embodiments, the spiral sheet terminii (e.g. 1622 and 1623) are constructed with a tapering geometry, such the their tips continue smoothly to the adjacent sheet surface.

In some embodiments, it is desired that the sleeve 1600 expand evenly along both the top and bottom ends of coil. This can be enabled by the use of tracking attachments such as 1641 and 1642, which, when placed at both ends of the coiled sleeve, keep the sleeve sheet moving parallel to the longitudinal axis of coiling throughout the sleeve expansion motion.

The pressure to cause the coiled sleeve to expand radially can optionally be exerted by inflation of the ETT cuff balloon.

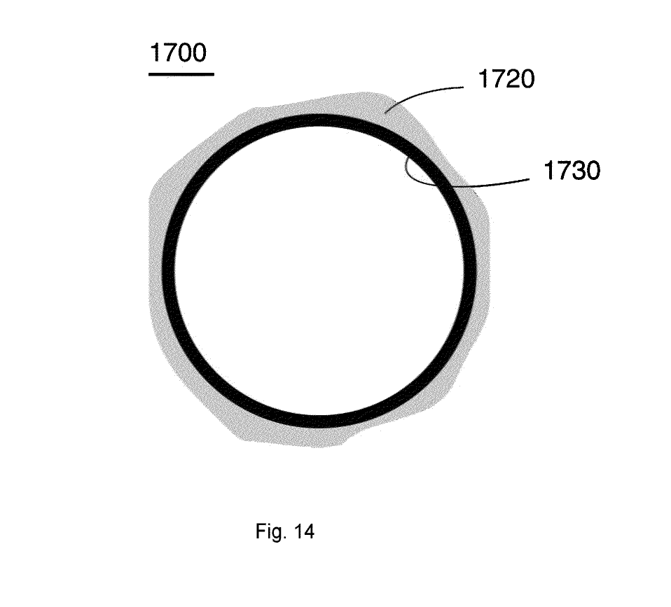

Reference is now made to FIG. 14. The trachea has the structure of a series of rings arranged one on top of the other, and, as explained earlier, has a cross-section which is not perfectly circular. Consequently, the trachea wall tissue has a non-uniform, wavy geometry along its length and a non-circular cross section. An expanding, rigidly flat sleeve will exert a non-uniform pressure along the trachea tissue. Thus, in some embodiments, in order to conform better to the trachea tissue, the sleeve has a compressible outer layer cover, which may be e.g. inflatable, or filled with a foam or gel material, or constructed from a deformable material.

FIG. 14 schematically shows a cross sectional view of a sleeve embodiment 1700, shown perpendicular to the longitudinal axis of the sleeve. The inner portion 1730 provides axial rigidity. However, unlike the sleeves discussed until now, sleeve 1700 also has an outer portion 1720, which is compressible and pliable along most of its surface area. Outer portion 1720 may be, for example, an inflatable bag, or it may be a thin, gel-filled bag. The pliable nature of outer portion 1720 enables it to conform to the trachea tissue surface. Provided that the total pressure exerted by the sleeve, including the outer outer portion thereof, on the trachea remains within the desired range, outer portion 1720 improves the sealing of the sleeve and the trachea in comparison to embodiments that lack the outer portion, and it functionally distributes more uniformly the pressure exerted by the sleeve on the non-uniform geometry of the trachea wall tissue. In some embodiments, inner and outer portions 1720 and 1730 are attached to one another, e.g. by gluing or ultrasonic welding. In other embodiments, the portions 1720 and 1730 are only attached to each other in some locations, but are not glued or connected over the full contact surface between them. When the outer layer 1720 is inflatable, means for inflating 1720, similar to those used for inflating the ETT cuff balloon, are provided. In other embodiments, outer portion 1720 is made of a compressible foam or rubber material.

A typical ETT tube has a well-defined directionality for placement into a human trachea. In particular, ETT tubes are axially arched towards the frontal side of the patient. ETT cuff balloons presently in use are circularly symmetric, even though the cross-section of the human trachea is not circularly symmetric. In particular, while the anterior side of the trachea has a semicircular arc shape delineated by cartilage, the posterior side of the trachea may be quite flat and even bulge in toward the trachea center. In order to meet the demands of conforming to such asymmetric conformations, in some embodiments the sleeve is made so that its radial expansion properties are asymmetrical, e.g. so that the outer surface thereof expands more easily toward one side of the trachea wall (the posterior side) than toward the opposite side. One illustrative technical realization of such an asymmetric sleeve can be obtained in an expandable sleeve where its outer layer is constructed with non-uniform thickness. In particular, when the outer layer is made of an elastic material (such as polyurethane, latex, or butyl rubber), it is known in the art that the coefficient of elasticity of such a layer is dependent on the thickness thereof; a thicker layer is stiffer than a thinner one made of the same material. Therefore, in some embodiments, the outer layer of an expandable sleeve can be made thicker on one side and thinner on the other side. Such a sleeve, when expanded, will have expanded more along its thin side than its thick side. Alternatively, utilization of the embodiment depicted in FIG. 14 may facilitate a better fit to asymmetric regions. It will also be appreciated that asymmetric sleeves transmit varying contact pressures to the trachea surface tissue.

Reference is now made to FIG. 8, which illustrates embodiments of the present invention. FIG. 8 depicts a configuration similar to that shown in FIGS. 2b and 9b, but here the thickness of the sleeve 820 is non-uniform. In particular, sleeve 820 is thin near its ends and thicker in the middle section. The thickness increase towards the center is preferably increased gradually, such that a smooth slant is created. Consequently, fluids 410 moving down the trachea collect near the ETT 860 and away from the trachea tissue 210.

It will be appreciated that in embodiments of the present invention such as are depicted in FIGS. 2b, 8 and 9b, the ETT cuff balloon 850, when fully inflated, has a diameter smaller than the trachea diameter. Instead, cuff 850 is inflated so that the diameter thereof fits the diameter 2Rᵢₙ of the inner surface 830 of sleeve 820 at its mid-section.

In accordance with some embodiments, sleeve 820 may be constructed so that sleeve 820 is itself composed of two concentric expanding sub-sleeves. For example, one sub-sleeve may delimit the inner surface 830, while the second sub-sleeve defines the outer surface which contacts, and preferable fits to the curvature of, the trachea wall tissue. The volume between these first and second sub-sleeves may be constituted from, for example, an inflatable membrane or an expanding gel or foam material, to give the full form and geometry of the sleeve 820.

In some embodiments, the sleeve (such as sleeve 820) is initially positioned in the trachea in a compressed or otherwise unexpanded state. Upon reaching the desired location, the sleeve is caused to expand until its outer surface presses against the trachea wall tissue 210. The expanded sleeve 820 is set so that over time the pressure it exerts on the trachea tissue 210 is less than 25 mm Hg. In addition to expansion via the cuff balloon, as shown, means of expanding the sleeve may constitute fluid or gas inflation means, expanding rings, or by analogy to methods known in the art of stent expansion.

Because the construction employed, the ETT cuff balloon 850 may be inflated, independently of the sleeve 820, to a pressure higher than 25 mm Hg against the inner surface 830 of sleeve 820.

In some embodiments, the sleeve, such as sleeve 820, may include antibacterial elements, such as an antibacterial coating, or an antibacterial drug(s) that is slowly released from the sleeve.

It will be appreciated that although, in accordance with embodiments of the present invention, the ETT cuff balloon can be inflated to a steady high pressure, without risk of tissue anoxia and other related complications in the tracheal tissue. However, in some embodiments, the pressure of the sleeve on the trachea wall tissue may be dynamically regulated independently from the ETT cuff.

In some embodiments, the sleeve element incorporates a re-collapse mechanism in order to enable its extraction at the end of intubation treatment. This may take the form, for example, of the sleeve being biased toward the collapsed state, such that when the cuff balloon is deflated, the sleeve collapses (as discussed above); other illustrative embodiments include the use of shape memory material in the formation of the sleeve, which facilitates the return of the sleeve to a collapsed state; or a thermoplastic resin that is worked in such a way that upon heating, the sleeve collapses. Such technologies per se are known in the art and have been used in context of stents, and are not described in detail here.

In accordance with some embodiments, the sleeve may be incorporated for delivery on top of the ETT itself. In particular, the sleeve may be expanded, at least partially, by inflation of the ETT cuff itself. Variations of such embodiments include direct attachment of the sleeve to the ETT cuff balloon along a circumference of the balloon. Such an arrangement improves the sealing between the balloon and renders the sealing independent of pressure.

Alternatively, in some embodiments, the sleeve may be placed in the trachea prior to the insertion of the ETT, using means analogous to those used in the art of stent delivery and placement. The ETT may be emplaced thereafter, in a manner that facilitates creation of a seal between the ETT and sleeve, for example by positioning the cuff balloon so that when inflated, it contacts the inner surface of the sleeve.

As discussed earlier, movement of the ETT cuff balloon in the trachea is a contributing factor to both leak of fluids and damage to the trachea tissue in presently-used ETTs. Due to head movement or tongue pressure, the upper portion of the tube is pushed and pulled upon by the patient at various times. In ETTs currently in use, this causes repeated movements of the cuff balloon along the trachea. The balloon thus rubs on the trachea tissue and slides over the lining fluids. Presented herein are alternative embodiments which reduce such movement. Referring now to FIGS. 15a and 15b, a sleeve 1920 is connected to ETT 260 via a thin, flexible membrane 1925, which is attached, e.g. by gluing or ultrasonic welding, to both the ETT along an outer circumference thereof and to the sleeve along an inner circumference thereof. Membrane 1925 may also be formed integerally with the ETT and/or the sleeve. Because membrane 1925 is easily deformable, in principle a significant freedom of up/down movement of the tube 260 relative to the trachea wall 210 and relative to sleeve 1920, without concomitant movement of sleeve 1920, is enabled, depending on the length of membrane 1925. Such relative movement is illustratively represented by the difference between FIGS. 15a and 15b, wherein the upward movement of the tube 260 has caused a change in the rest shape of the connecting membrane 1925 but without dragging up the sleeve 1920. We note that the membrane 1925 may or may not itself be inflatable. Membrane 1925 may have any suitable shape that allows movement of the tube relative to the trachea wall and the sleeve, such as a disk, ring, or cone shape.

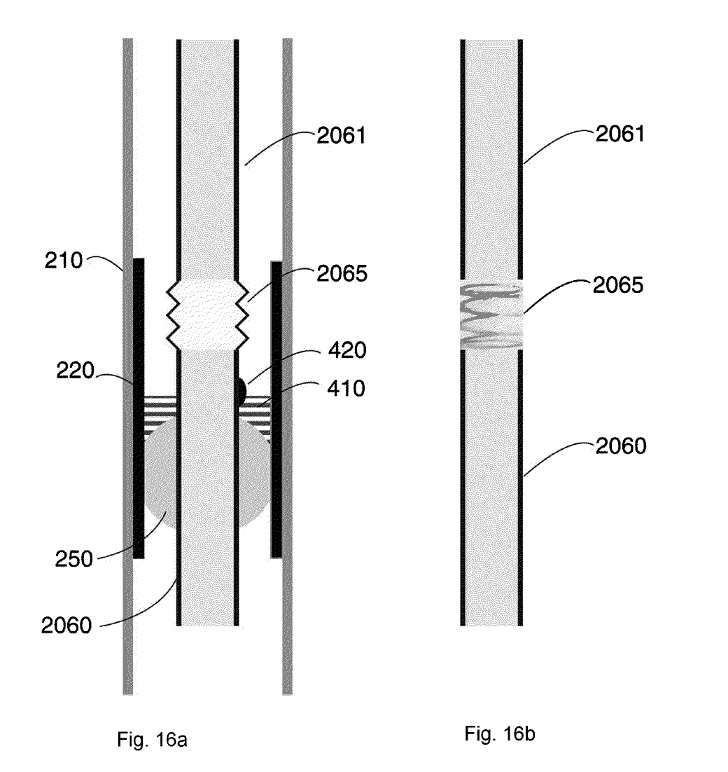

An alternative method of reducing the movement of the balloon and/or sleeve is to introduce a flexible connection between the top and bottom portions of the ETT. Reference is now made to FIG. 16a, which illustrates the principle according to which a flexible tube midsection 2065 (depicted in the drawing as an accordion-like part) is capable of stretching or expanding longitudinally. This allows the distance between upper tube section 2061 lower tube section 2060 to vary. Hence, forced movement of the top tube section 2061 will not necessarily force a movement of the bottom tube section to which the balloon and/or the sleeve are connected.

FIG. 16b illustrates an embodiment in which the flexible mid section 2065 is formed from a spring-like skeleton which is wrapped within an impermeable but stretchable membrane.

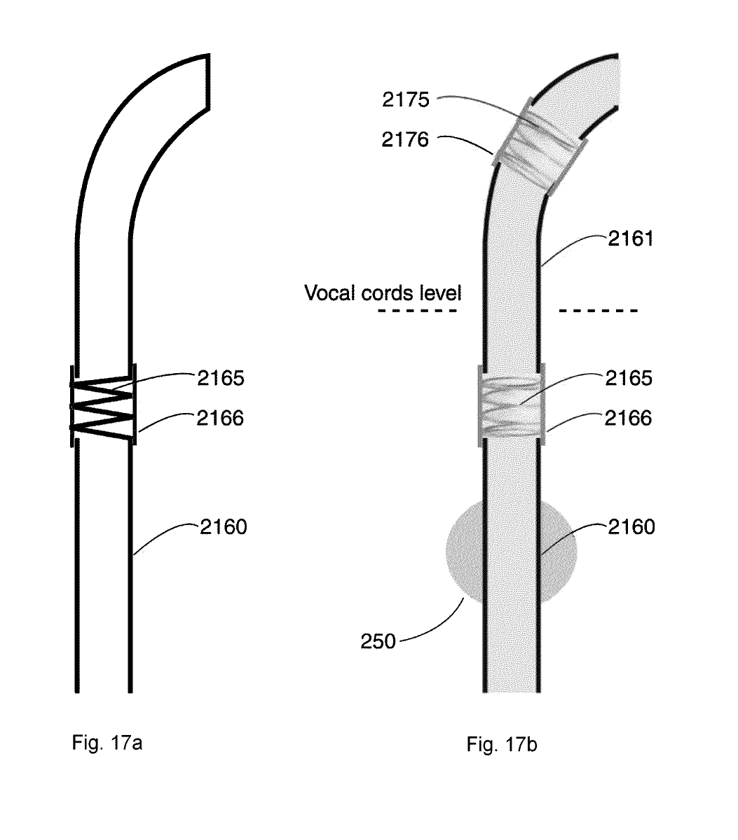

FIG. 17a illustrates an embodiment in which a flexible mid-section 2165 is placed below the vocal cords and/or a flexible mid-section 2175 is placed above the vocal cords. Either of these midsections can be formed from a spring-like skeleton which is wrapped within an impermeable membrane; such membranes are indicated by elements 2166 and 2176, respectively, in FIG. 17a.

FIG. 17b illustrates an embodiment in which flexible mid section 2165 formed via a helical cut in a regular tube wall, i.e. spring-like element 2165 is made of the same material as the ETT itself, and there is an unbroken continuation of the tube material between the lower section 2160 and upper section 2161 through the springy mid-section 2165, without any additional bonding material.

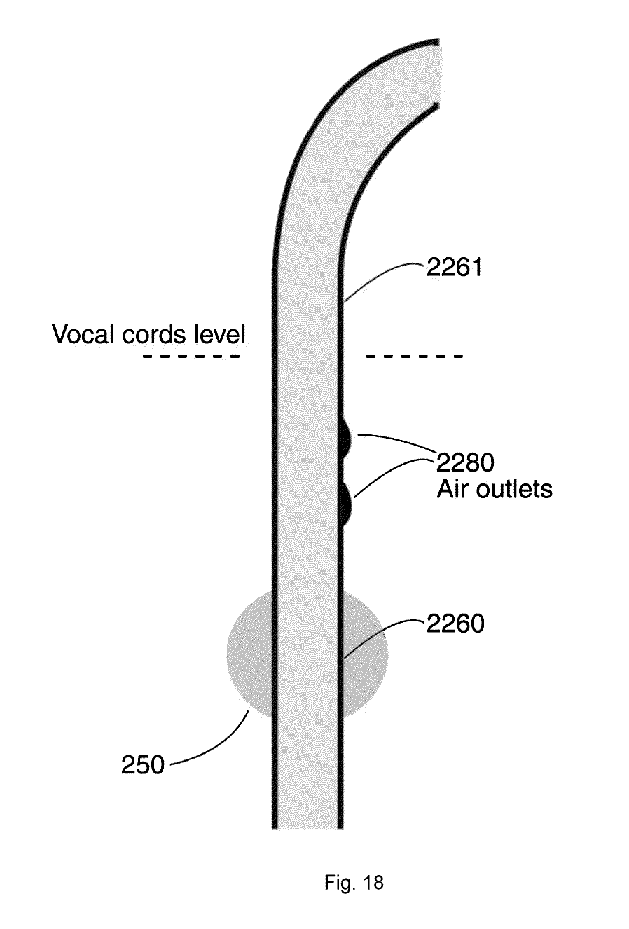

Reference is now made to FIG. 18. One of the reasons for increased levels of VAP after 72 h of intubation is the change in oral flora, which results, at least in part, from the lack of air movement through the oral cavity. FIG. 18 illustrates an ETT embodiment with air outlets 2280 positioned on the side of the tube. Outlets 2280 are preferably placed below the vocal cords. The structure of outlets 2280 structure is reminiscent of the structure suction inlets 420, and can be similarly fed by a secondary lumen embedded in the ETT wall (not shown but known in the art with respect to suction inlets). One or more outlets 2280 can be implemented. In contrast to suction inlets, air is not sucked into the outlet 2280 but instead is blown out into the trachea lumen. In some embodiments, the timing of air blowing through outlets 2280 is correlated with the rhythm of air inhalation/exhalation ventilation of the patient. Preferably, the blowing of air from outlets is 2280 is correlated with the peak of ventilation positive pressure.

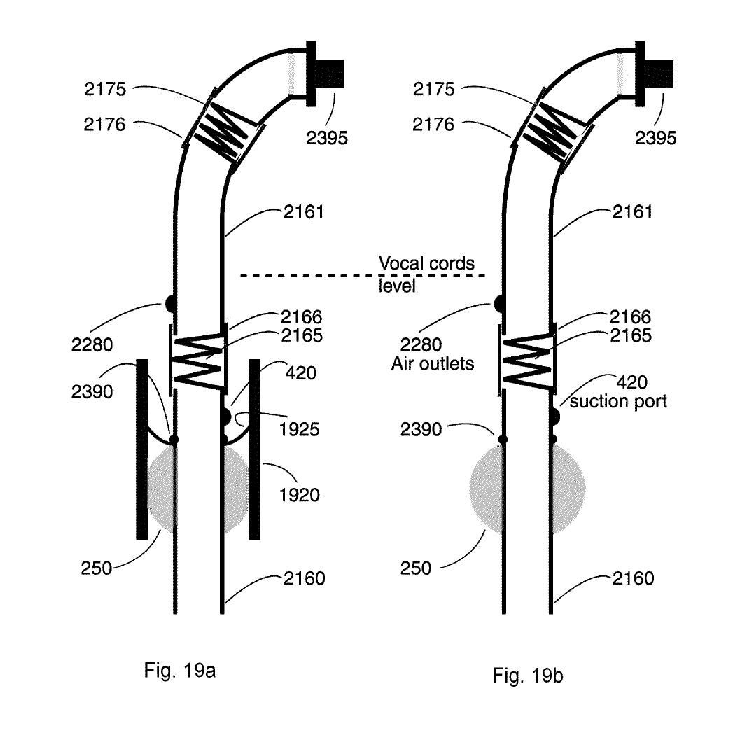

It will be appreciated that various combinations and subcombinations of features described above may be implemented in accordance with embodiments of the invention. For example, while in general use of a sleeve is contemplated, the use of a flexible connecting member between upper and lower portions of the ETT, such as is depicted in FIGS. 16 and 17, or the use of air outlets as in FIG. 18, may be implemented with balloon cuff ETTs currently in use, even without the addition of a sleeve. FIG. 19 illustrates an intubation system in accordance with embodiments of the present invention, that implements a particular combination of features. The general approach is to have an option to intubate a patient first with a conventional looking ETT with cuff balloon only (FIG. 19a), and then in an independent treatment step to add a sleeve element (FIG. 19b). Such an arrangement is useful, for example, where intubation is necessary, but it is unknown at the time if such intubation will be short-term or long-term.

In the system shown in FIG. 19, it is possible to detach and reconnect the top end 2395 of the ETT. The patient is first intubated with a conventional cuff balloon ETT, viz. without the sleeve (e.g., during surgery), and a conventional cuff 250 is inflated to a functional level, as is known in the art. When the patient is brought into the ICU, there is an option to insert sleeve 1920 into the trachea, over balloon cuff 250. In order to emplace sleeve 1920 in an already-intubated patient, the detachable top 2395 is quickly opened, the sleeve is slid over the top of the ETT, and the top 2395 is immediately reconnected in order to continue normal ventilation of the patient. Sleeve 1920 is initially delivered in a compressed configuration of small diameter around the top of the ETT tube. The sleeve is then lowered down the ETT tube to its designated sealing location below the vocal cords. The cuff 250 is decompressed in order to allow the lowering of the sleeve into its final position. In some embodiments, there is an indentation or a protrusion 2390 (above the cuff 250) which locks into position a membrane 1925 which is connected to sleeve 1920. The sleeve is expanded (for example, inflated with gas) to snugly press against the trachea wall tissue. Optionally, as illustrated in FIG. 19b, the cuff balloon 250 is re-inflated just to assist in maintaining the shape of the sleeve 1920. In some embodiments, as illustrated in FIGS. 19a and 19b, the ETT system may incorporate a suction port 420 and/or air outlet ports 2280. In some embodiments, as illustrated in FIGS. 19a and 19b, the ETT system may incorporate dynamic length tube sections 2175 above the vocal cords, and/or 2165 below the vocal cords.

It will be appreciated that although the drawings depict the construction and use of an endotracheal tube, many embodiments of the present invention are equally applicable in the case of a tracheotomoty or tracheostomy tube, and are thus encompassed within the scope of this application. Furthermore, for the sake of simplicity, in the specification above and in the claims that follow, the term “tracheal tube” will be understood as encompassing endotracheal tubes as well as tracheotomy and tracheostomy tubes, and “tracheal intubation” will be understood as encompassing intubation in the trachea using such tubes.

The variations described above with respect to methods and apparatus/systems in accordance with embodiments of the invention are applicable, mutatis mutandis, with respect to kits in accordance with embodiments of the invention.

Various embodiments of the invention have been described in detail, but it will be understood by those skilled in the art that variations and modifications can be effected within the spirit and scope of the invention.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the general combination of parts that perform the same functions as exemplified in the embodiments, and includes both combinations and sub-combinations of the various features described hereinabove as well as variations and modifications thereof, which would occur to persons skilled in the art upon reading the foregoing description.

1. A method for tracheal intubating in a patient in need thereof, comprising aligning, in the trachea of said patient,
(a) a tracheal tube having a distal end which is inserted into the trachea and a proximal end which remains outside the trachea;
(b) a sleeve through which said tracheal tube passes, said sleeve having an inner surface which faces said tracheal tube and an outer surface and proximal and distal ends which correspond to the proximal and distal ends, respectively, of said tracheal tube, the sleeve being of a length less than the distance between the larynx and the carina; and
(c) a cuff which contacts at least a portion of the outer surface of said tracheal tube and at least a portion of said inner surface of said sleeve
in a manner which substantially creates a seal between the distal and proximal ends of the inner surface of said sleeve and the distal and proximal ends of the outer surface of said tracheal tube, whereby to radially space the tracheal tube from the sleeve.

2. A method according to claim 1, wherein the outer surface of said sleeve sealingly contacts the wall of said trachea.

3. A method according to claim 1, wherein said tube is inserted into said trachea before the sleeve is inserted into the trachea.

4. A method according to claim 1, wherein said tube is inserted into said trachea after the sleeve is inserted into the trachea.

5. A method according to claim 1, wherein said sleeve and said tube are inserted concomitantly into the trachea.

6. A method of reducing the likelihood of fluids leaking from the trachea of a patient undergoing intubation into a lung theorof, the method comprising deploying a tracheal tube, a sleeve and a cuff into a human trachea such that after deployment, the tracheal tube passes through the sleeve within the trachea, the cuff contacts the outer surface of the tracheal tube and the inner surface of the sleeve and spaces the sleeve from the tube, and the outer surface of the sleeve contacts the trachea, so as to provide a seal, in the interstitial area between the wall of the trachea and the tube, between a proximal portion of the human trachea above the cuff and a distal portion of the human trachea below the cuff.

7. The method of claim 6 wherein the cuff is an expandable cuff, the sleeve is longitudinally rigid and radially expandable along at least a portion thereof, and the deploying includes:
(a) simultaneously inserting the tracheal tube, the expandable cuff and the sleeve into the trachea; and
(b) after the inserting, inflating the inflatable cuff,
so as to cause radial expansion of the sleeve and create said seal by contacting the sleeve to an inner surface of the trachea and by sealingly contacting the inner surface of the sleeve and the outer surface of the tube.

8. The method of claim 6 wherein the cuff is an expandable cuff, the sleeve is longitudinally rigid and radially expandable along at least a portion thereof, and the deploying includes:
(a) emplacing the sleeve in the trachea of the patient;
(b) thereafter inserting the tube and the cuff into trachea such that the tube passes through the sleeve with cuff positioned between the tube and sleeve; and
(c) thereafter expanding the cuff so that the outer surface of the sleeve contacts the wall of the trachea, the cuff contacts the inner surface of the sleeve and the outer surface of the tube and spaces the sleeve from the tube, whereby to provide said seal.

9. The method of claim 6 wherein the cuff is an expandable cuff, the sleeve is longitudinally rigid and radially expandable along at least a portion thereof, and the deploying includes:
(a) inserting the tube and the cuff into trachea;
(b) emplacing the sleeve in the trachea of the patient such that the tube passes through the sleeve with cuff positioned between the tube and sleeve; and
(c) thereafter expanding the cuff so that the outer surface of the sleeve contacts the wall of the trachea, the cuff contacts the inner surface of the sleeve and the outer surface of the tube and spaces the sleeve from the tube, whereby to provide said seal.

10. A method according to claim 9, wherein the sleeve is sized and shaped to contact the tracheal wall.

11. A method according to according to claim 10 wherein the sleeve has a substantially circular cross-section.

12. A method according to according to claim 11, wherein the maximum diameter of the sleeve is not more than about 2.5 cm.

13. A method according to claim 10 wherein the sleeve has a non-circular cross section.

14. A method according to claim 10, wherein the sleeve is sized and shaped to contact the tracheal wall in a manner which substantially seals the lungs from the pharynx.

15. A method according to claim 10, wherein said sleeve is of a length that fits between the larynx and the carina.

16. A method according to claim 15, wherein said sleeve has a length of from 2 to 8 cm.

17. A method according to claim 16, wherein the distal end of the sleeve is positioned 2 to 6 cm above the carina.

18. A method according to claim 17, wherein the sleeve is substantially rigid in its axial direction along at least 50% of its length.

19. A method according to claim 18, wherein the sleeve is substantially rigid in its axial direction along at least 60% of its length.

20. A method according to claim 19, wherein the sleeve is substantially rigid in its axial direction along at least 70% of its length.

21-197. (canceled)
